(19) United States
(12) Patent Application Publication
(10) Pub. No.: US 2012/0316482 A1
(43) Pub. Date: Dec. 13, 2012

(54) ORTHOTIC BONE STIMULATOR

(76) Inventor: Aftab Karim, Burton, MI (US)

(21) Appl. No.: 13/445,697

(22) Filed: Apr. 12, 2012

Related U.S. Application Data
(60) Provisional application No. 61/475,105, filed on Apr. 13, 2011.

(51) Int. Cl. A61N 1/00; A61F 5/00

(52) U.S. Cl. 602/5; 607/51

(57) ABSTRACT

The invention relates to an orthotic bone stimulator and the methods of use pertaining thereto.

### CROSS-REFERENCE TO RELATED APPLICATIONS

This application claims priority under 35 U.S.C. §119(e) to U.S. Provisional Patent Application Ser. No. 61/475,105, filed on Apr. 13, 2011, which application is incorporated by reference herein in its entirety.

### FIELD OF THE INVENTION

The invention relates an orthotic bone stimulator to and the methods pertaining thereto.

### BACKGROUND OF THE INVENTION

Fractures, or broken bones, are common injuries that can take months or even years to fully heal. The healing process is generally the same for all fractures; through a series of stages, new bone forms and fills in the fractured area. The rate of healing and the ability to remodel a fractured bone vary tremendously for each person and, in general, depend on several factors, such as age, overall state of health, the type of fracture, and the bone involved. Specifically, smoking, diabetes, obesity, and advanced age can increase the difficulty of fracture healing due in part to diminished circulation, and other factors not well understood.

The use of electrical stimulation to improve the effectiveness of fracture healing has grown significantly over the past decade. Electrical or ultrasound stimulation is a good option for patients who have bone healing problems, or fractures that have poor healing potential. As the number of scientific and clinical studies validating the use of electrical or ultrasound stimulation to enhance spine fusion has increased, there is a better understanding among spine surgeons about how and when to use specific electrical stimulation devices to aid in the healing of spine fusion.

Some of the problems associated with this type of treatment includes patient compliance or cooperation, and accuracy in the placement of the simulator. Typical treatment regimens include applying the bone growth stimulator to the fracture for about 20 minutes to up to 4 hours per day in order to provide a benefit. In addition, the placement of the stimulators much be such that the bone is sufficiently stimulated.

One way these problems can be avoided is with the use of an invasive or semi-invasive electrical bone growth stimulator, which is implanted at the non-healing fracture or bone fusion site and delivers electrical energy directly to the site. However, invasive electrical bone growth stimulators involve threading the cathode through or around the fractured bone with the anode and power supply implanted in the surrounding soft tissue. Semi-invasive electrical stimulators use a cathode implanted in the cortex of one end of the nonunion site and attached to an external anode and power supply. Accordingly, both types require two surgeries for the patient to undergo; one to implant the device, and one to remove it.

The device disclosed herein provides for a non-invasive stimulation which provides for an accurate, circumferential and uniform bone stimulation which is not provided with the use of known devices.

### BRIEF SUMMARY OF THE INVENTION

The present invention is predicated on the surprising and unexpected discovery that bone fractures and discontinuities can be more efficiently healed with the use of an orthotic bone stimulator which comprises a bone growth stimulator attached to a stabilization device, such that the bone growth stimulator can be positioned directly adjacent to the fractured bone. The bone growth stimulator and stabilization device are joined or fastened together such that a limb or part of the body of a patient which comprises the fractured bone is sufficiently immobilized and the bone growth stimulator is secured in place. The orthotic bone stimulator disclosed herein provides superior results when compared to the use of the separate components together.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows a preoperative CT scan of the cervical spine of a patent in sagittal view (March 2009).

FIG. 2 shows a postoperative (2 months) CT scan of the cervical spine in sagittal view (May 2009) after the patient was stabilized with an occipital cervical fusion plus treated with the orthotic bone stimulator disclosed herein.

FIG. 3 shows an exemplary orthotic bone stimulator for the torso, where the electrical stimulator portion of the device is shaded.

FIG. 4 shows an exemplary orthotic bone stimulator for the cervical region, where the electrical stimulator portion of the device is shaded.

### DETAILED DESCRIPTION OF THE INVENTION

Before the present compositions and methods are described, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to a “fracture” includes a plurality of fractures.

### 1. Definitions

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5% or 1%.

The term “fractured bone” is intended to refer to a medical condition in which there is a break or discontinuity of the bone. The bone fracture can be the result of high force impact or stress, such as a compression fracture, or trivial injury as a result of certain medical conditions that weaken the bones, such as osteoporosis, bone cancer, or osteogenesis imperfecta, known as a pathological fracture. Also, the bone fracture can be the result of a surgical procedure which causes a discontinuity of the bone, or natural discontinuity, such as spinal fusion. The type of bone fracture can be simple or multifragmentary, and can be closed or open. Furthermore, the fracture can be transverse, oblique, spiral, or segmental.

The term “bone growth stimulator” is intended to refer to a device that stimulates bone growth. Exemplary bone growth stimulators which can be used in the devices disclosed herein employ electronic or ultrasound stimulation to facilitate bone growth. However, the method of stimulation is not imperative to the devices or methods disclosed herein, and as such, other methods of stimulation are contemplated. The bone growth stimulators of the present invention are preferably noninvasive.

The term “stabilization device” is intended to refer to a removable orthotic device which immobilizes or limits the mobility of a limb, neck or the torso of a patient.

The term “attached” is intended to refer to the bone growth stimulator and the stabilization device being joined or fastened together. In some embodiments, the bone growth stimulator and the stabilization device are removably attached using a fastener, such as clips, snaps, Velcro®, etc., such that the bone growth stimulator and the stabilization device can be removed or adjusted. In some embodiments, the bone growth stimulator and the stabilization device are permanently attached using brackets, stitching, etc. In general, the leads of the bone growth stimulator are incorporated into the stabilization device.

The phrase “directly adjacent to the fractured bone” is intended to refer to the placement of the electrical or ultrasound stimulating portion of the bone growth stimulator such that the fractured bone is in sufficiently close proximity to be affected by the stimulation (i.e. electrical, ultrasound, etc.).

The phrase “applying the bone growth stimulator to the fractured bone” is intended to refer to powering or turning on the growth stimulator such that the electrical or ultrasound stimulation, or other method of stimulation, affects the fractured bone such that the bone is substantially or completely healed (promoted ossification).

The term “patient” is intended to refer to, without limitation, a human, domestic animal (i.e., dog or cat), farm animal (i.e., pig, cow, horse, or pig), or laboratory animal (i.e., mouse, rat, hamster, guinea pig, rabbit, dog, or monkey).

### 2. Device of the Invention

A bone growth stimulator is a device that employs either electronic stimulation or ultrasound to facilitate bone growth and therefore heal fractured bones. Bone growth stimulators are typically used when a bone does not heal on its own even after an extended amount of time, usually about 3 to 9 months. Bone growth stimulators are indicated for use only in individuals who are skeletally mature, or when all bone growth is complete; the cartilage cells of the growth plate cease to proliferate, the growth plate becomes thinner, is replaced by bone and disappears, and the epiphysis is “closed” or fused with the shaft.

The present invention is predicated on the discovery that a bone fracture can be healed or re-calcified to greater degree with the present device as compared to an orthotic device with a separate, unattached bone stimulator. Whereas patient compliance would likely increase with the use of the orthotic bone stimulator disclosed herein, it is not believed to be the reason for the surprising discovery that such an enhanced degree of healing of bone fractures is observed.

In one embodiment, the present invention is directed to an orthotic bone stimulator for treatment of a fractured bone in a patient comprising a bone growth stimulator attached to a stabilization device such that the bone growth stimulator can be positioned directly adjacent to the fractured bone.

In some embodiments, the bone growth stimulator is an ultrasonic bone stimulator. Ultrasound bone growth stimulators are noninvasive and are designed to transmit low-density, pulsed, high-frequency acoustic pressure waves to accelerate healing of fresh fractures and to promote healing of delayed unions and nonunions that are not sufficiently healed under standard treatment methods. Ultrasound devices have been proven to stimulate fresh fracture healing and healing of nonunions in humans. Such devices have also been suggested to enhance healing of fractures that occur in patients with diseases such as diabetes, vascular insufficiency, and osteoporosis, and those taking medications such as steroids, non-steroidal anti-inflammatory drugs (NSAIDs), or calcium channel blockers. Although the exact mechanism for fracture healing is unclear, it is thought that ultrasound causes biochemical changes at the cellular level to accelerate bone formation. It has been hypothesized that ultrasound increases blood flow to the capillaries, enhancing cellular interaction. Such stimulators are commercially available, such as, but not limited to, the EXOGEN™ Ultrasound Bone Healing System and Sonic Accelerated Fracture Healing System (SAFHS®) (both available from Smith & Nephew).

In some embodiments, the bone growth stimulator is an electrical bone stimulator, also referred to herein as an electronic or electric bone stimulator. Bone fixation devices, such as screws, rods, pins, plates, etc., made from magnetic materials may compromise the effects of electric bone growth stimulators. Typical electrical bone growth stimulators fall into one of three categories: noninvasive, semi-invasive or invasive. However, the bone growth stimulators of the device disclosed herein are noninvasive, while providing benefits which exceed the use of both semi-invasive and invasive. Noninvasive electrical bone growth stimulators use inductive and conductive methods to deliver a broad, uniform electric field, pulse electromagnetic field (PEMF), or combined electromagnetic (CMF) field to the fracture site via treatment coils or disks, which are typically placed on the skin and attached to an external power supply. Contrarily, in the devices disclosed herein, the treatment coils or disks are attached to the stabilization device such that the treatment coils or disks are positioned adjacent to the bone fracture. Direct electrical current has been shown to have a stimulatory effect on bone formation. Such stimulators are commercially available, such as, but not limited to, OL 1000® and SpinaLogic Bone Growth Stimulator® (Regentek, formerly OrthoLogic, Tempe, Ariz.); Physio-Stim Lite®, Spinal-Stim Lite® (Orthofix, Inc., Richardson, Tex.); EBI Bone Healing System®, SpinalPak®, and OrthoPak® (Biolectron, a subsidiary of Electro-Biology, Inc., Parsippany, N.J.).

The bone fracture can be the result of high force impact or stress, such as a compression fracture, or trivial injury as a result of certain medical conditions that weaken the bones, such as osteoporosis, bone cancer, or osteogenesis imperfecta, known as a pathological fracture. Also, the bone fracture can be the result of a surgical procedure which causes a discontinuity of the bone, or natural discontinuity, such as spinal fusion. Various types of bone fractures can be treated with the device disclosed herein, including, simple or multifragmentary, and can be closed or open. Furthermore, the bone fracture can be transverse, oblique, spiral, or segmental.

It is contemplated that the fractured bone can be any bone, including one or more bones selected from the group consisting of vertebrae, skull, rib, fibula, tibia, femur, scaphoid, metacarpal, metatarsal, humerus, ulna, radius and pelvis. Accordingly, the appropriate stabilization device should be used to limit the mobility of the fracture site. Suitable stabilization devices are known in the art, and include by way of example, a halo, cervical collar, thoracic orthosis, lumbosacral orthosis, thoracolumbar orthosis, and peripheral extremity orthosis (i.e. a splint or cast).

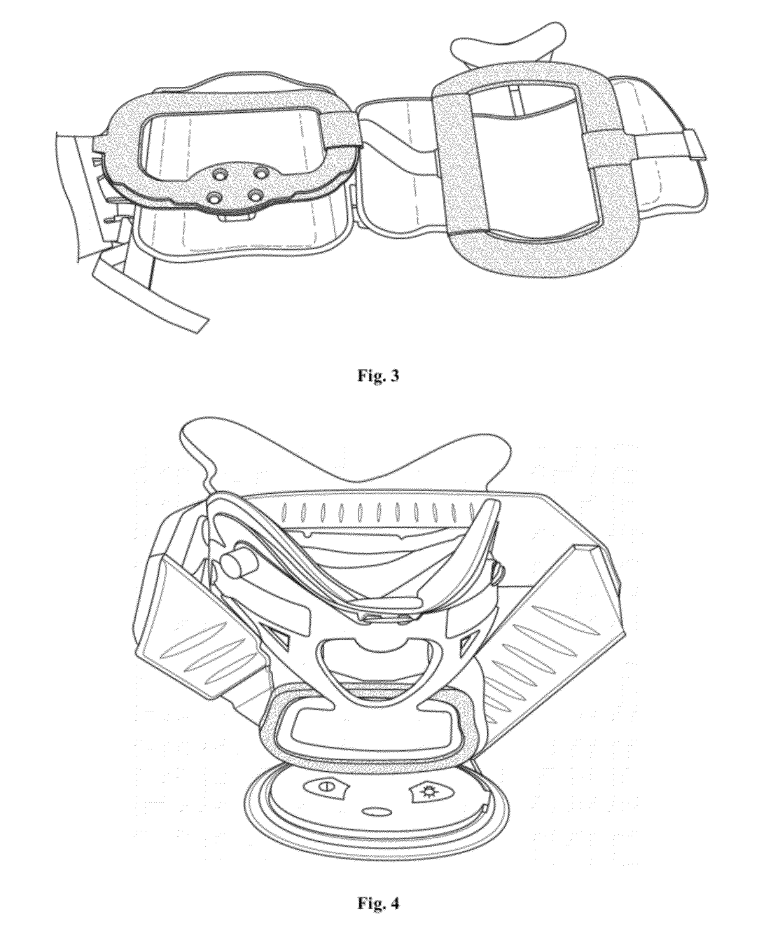

In certain embodiments, the fractured bone is one or more vertebrae. The bone fracture can be the result of a spinal fusion or can be the result of a compression fracture. The bone stimulator can be attached to the stabilization device along the contours of the orthosis to permit circumferential and uniform bone stimulation. Accordingly, the propensity to fuse a fracture is enhanced. Such stabilization devices include halos, cervical collars, thoracic orthosis, lumbosacral orthosis, thoracolumbar orthosis and peripheral extremity orthosis. In one embodiment, the one or more vertebrae include vertebrae in the cervical, lumbar or thoracic region. In one embodiment, the one or more vertebrae include vertebrae in the lumbar or thoracic region (see FIG. 3). In one embodiment, the one or more vertebrae include vertebrae in the cervical region (see FIG. 4).

In another embodiment, the present invention is directed to an orthotic bone stimulator for the prevention and/or treatment of osteoporotic bone loss in a patient, wherein said orthotic bone stimulator comprises a bone growth stimulator attached to a stabilization device such that the bone growth stimulator can be positioned directly adjacent to a desired treatment site.

It is contemplated that the desired treatment site can be any bone, including one or more bones selected from the group consisting of vertebrae, skull, rib, fibula, tibia, femur, scaphoid, metacarpal, metatarsal, humerus, ulna, radius and pelvis. Accordingly, the appropriate stabilization device should be used. Suitable stabilization devices are known in the art, and include by way of example, a halo, cervical collar, thoracic orthosis, lumbosacral orthosis, thoracolumbar orthosis, and peripheral extremity orthosis (i.e. a splint or cast).

3. Methods of the Invention

In one embodiment, the methods disclosed herein employ the device above for the treatment of bone fractures. Accordingly, disclosed herein is a method for treating a fractured bone in a patient comprising positioning an orthotic bone stimulator which comprises a bone growth stimulator attached to a stabilization device such that the bone growth stimulator can be positioned directly adjacent to the bone fracture of the patient and applying the bone growth stimulator to the fractured bone. It is contemplated that the fractured bone can be any bone, including one or more bones selected from the group consisting of vertebrae, skull, rib, fibula, tibia, femur, scaphoid, metacarpal, metatarsal, humerus, ulna, radius and pelvis. In one embodiment, the fractured bone is one or more vertebrae. In one embodiment, the one or more vertebrae include vertebrae in the cervical, lumbar or thoracic region. In one embodiment, the one or more vertebrae include vertebrae in the lumbar or thoracic region (see FIG. 3). In one embodiment, the one or more vertebrae include vertebrae in the cervical region (see FIG. 4).

In the methods disclosed herein, the orthotic bone stimulator is applied daily, preferably at prescribed intervals, until sufficient healing of the fractured bone occurs. The methods disclosed herein provide for non-invasive, consistent bone stimulation which heals fractured bones to a greater degree when compared to what would be expected with the use of a bone stimulator alone.

In some embodiments, the patient has poor bone healing. Patients who suffer from one or more of tabes dorsalis, peroneal muscular atrophy, malignant neoplasm, diabetes, obesity, alcohol-induced mental disorders, alcohol dependence syndrome, tobacco use disorder, syringomyelia, syringobulbia, renal disease, arthrodesis status, or a mechanical complication of internal orthopedic device, implant, or graft typically have poor bone quality and would benefit from the methods disclosed herein. In some embodiments, the mechanical complication of internal orthopedic device, implant, or graft is a spinal fusion failure.

In certain embodiments, the fractured bone is one or more vertebrae. Accordingly, the method can comprise treating a segment of the spine following, or to augment, spinal fusion. In addition, the bone fracture can be the result of a compression fracture. The bone stimulator can be attached to the stabilization device along the contours of the orthosis to permit circumferential and uniform bone stimulation. Accordingly, the methods disclosed herein can include stabilization devices such as halos, cervical collars, thoracic orthosis, lumbosacral orthosis, thoracolumbar orthosis and peripheral extremity orthosis.

In another embodiment, methods disclosed herein employ the device above for the prevention and/or treatment of osteoporotic bone loss in a patient. Osteoporosis is a disease which characterized by the thinning of bone tissue and loss of bone density over time. Patients suffering from the disease generally have low bone mass, increased bone fragility, and a greater risk for bone fracture. Current methods for the prevention and/or treatment of osteoporosis are often accompanied by undesirable side effects, such as inflammation of the esophagus, nausea, abdominal pain, osteonecrosis of the jaw, irregular heartbeats, and visual disturbances with the use of certain medicines, and the use of hormone therapy can increase your risk of blood clots, endometrial cancer, breast cancer and possibly heart disease. Therefore, therapies which can minimize damaging side effects are necessary.

Accordingly, disclosed herein is a method for the prevention and/or treatment of osteoporotic bone loss in a patient comprising positioning an orthotic bone stimulator which comprises a bone growth stimulator attached to a stabilization device such that the bone growth stimulator can be positioned directly adjacent to a desired treatment site of the patient and applying the bone growth stimulator to the desired treatment site. It is contemplated that the desired treatment site can be any bone, including one or more bones selected from the group consisting of vertebrae, skull, rib, fibula, tibia, femur, scaphoid, metacarpal, metatarsal, humerus, ulna, radius and pelvis. In one embodiment, the desired treatment site is the one or more vertebrae. In one embodiment, the one or more vertebrae include vertebrae in the cervical region. In one embodiment, the desired treatment site is the pelvis.

In such methods, the orthotic bone stimulator is applied between 4 to 7 days a week, preferably at prescribed intervals (e.g., 20 minutes per day). Such a treatment regimen can continue indefinitely for the prevention of bone loss, or can be continued until new bone formation is sufficiently achieved. It is contemplated that the methods disclosed herein provide for non-invasive, consistent bone stimulation which can prevent and/or treat osteoporotic bone loss in a patient to a greater degree when compared to what would be expected with the use of a bone stimulator alone.

### 4. EXAMPLES

### Example 1

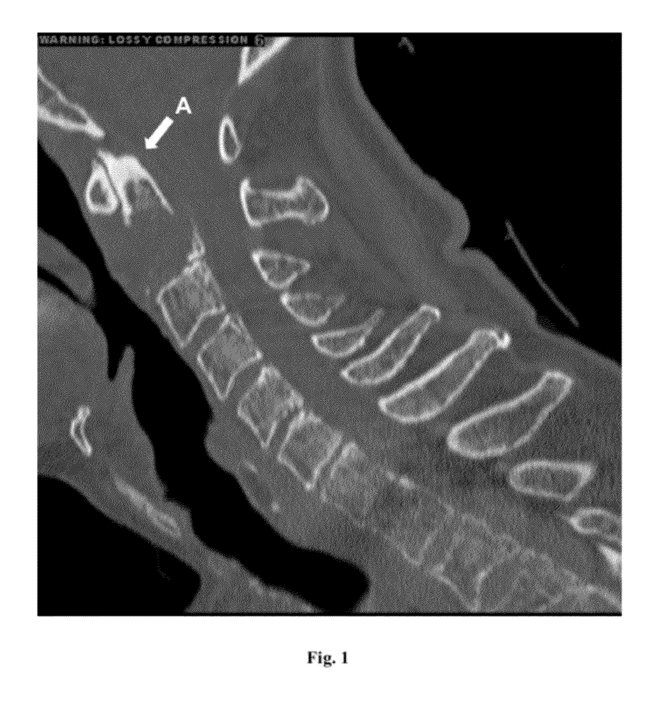

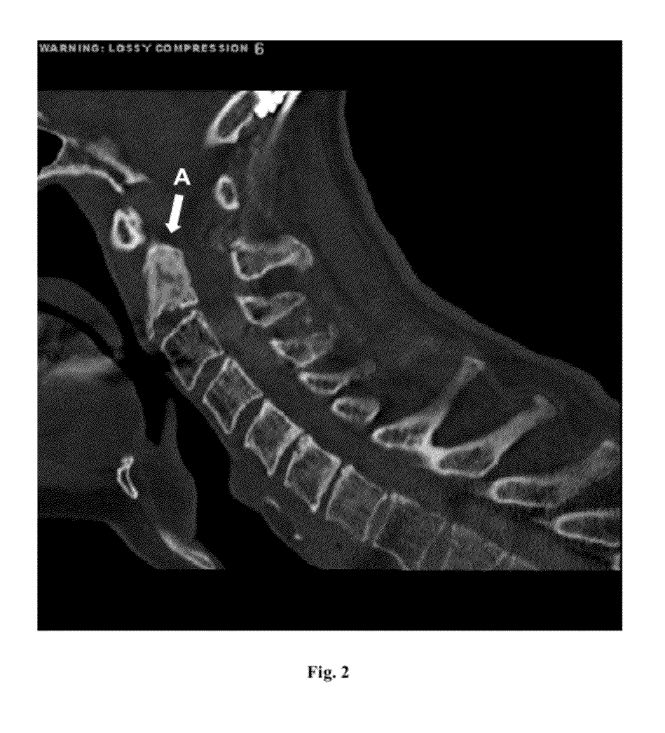

FIG. 1 represents the preoperative CT scan of the cervical spine in sagittal view (March 2009) of a human patient suffering from a fracture at C2. FIG. 2 shows the postoperative (2 months) CT scan of the cervical spine in sagittal view (May 2009) after the patient's spine was first stabilized with an occipital cervical fusion, followed by the use of an orthotic bone stimulator as disclosed herein in the form of a cervical collar. The superior degree of ossification of the C2 vertebral body as shown in FIG. 2 (at A) after only 2 months after the surgery is a result of the orthotic bone stimulator of the present invention.

It will be appreciated that those skilled in the art will be able to devise various arrangements which, although not explicitly described or shown herein, embody the principles of the invention and are included within its spirit and scope. Furthermore, all conditional language recited herein is principally intended to aid the reader in understanding the principles of the invention and the concepts contributed by the inventors to furthering the art, and are to be construed as being without limitation to such specifically recited conditions. Moreover, all statements herein reciting principles, aspects, and embodiments of the invention are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An orthotic bone stimulator for treatment of a fractured bone in a patient comprising a bone growth stimulator attached to a stabilization device such that the bone growth stimulator can be positioned directly adjacent to the fractured bone.

2. The orthotic bone stimulator of claim 1, wherein the fractured bone is one or more bones selected from the group consisting of vertebrae, skull, rib, fibula, tibia, femur, scaphoid, metacarpal, metatarsal, humerus, ulna, radius and pelvis.

3. The orthotic bone stimulator of claim 1, wherein the fractured bone is one or more vertebrae.

4. The orthotic bone stimulator of claim 3, wherein the one or more vertebrae are in the cervical region.

5. The orthotic bone stimulator of claim 1, wherein the stabilization device is a device for treating a compression fracture.

6. The orthotic bone stimulator of claim 1, wherein the stabilization device is selected from the group consisting of a halo, cervical collar, thoracic orthosis, lumbosacral orthosis, thoracolumbar orthosis, and peripheral extremity orthosis.

7. The orthotic bone stimulator of claim 1, wherein the bone growth stimulator is an ultrasonic bone stimulator.

8. The orthotic bone stimulator of claim 1, wherein the bone growth stimulator is an electrical bone stimulator.

9. An orthotic bone stimulator for the prevention and/or treatment of osteoporotic bone loss in a patient, wherein said orthotic bone stimulator comprises a bone growth stimulator attached to a stabilization device such that the bone growth stimulator can be positioned directly adjacent to a desired treatment site.

10. The orthotic bone stimulator of claim 9, wherein the desired treatment site is one or more bones selected from the group consisting of vertebrae, skull, rib, fibula, tibia, femur, scaphoid, metacarpal, metatarsal, humerus, ulna, radius and pelvis.

11. The orthotic bone stimulator of claim 10, wherein the desired treatment site is one or more vertebrae.

12. The orthotic bone stimulator of claim 11, wherein the one or more vertebrae are in the cervical region.

13. The orthotic bone stimulator of claim 10, wherein the desired treatment site is the pelvis.

14. The orthotic bone stimulator of claim 9, wherein the stabilization device is selected from the group consisting of a halo, cervical collar, thoracic orthosis, lumbosacral orthosis, thoracolumbar orthosis, and peripheral extremity orthosis.

15. The orthotic bone stimulator of claim 9, wherein the bone growth stimulator is an ultrasonic bone stimulator.

16. The orthotic bone stimulator of claim 9, wherein the bone growth stimulator is an electrical bone stimulator.

17. A method for treating a fractured bone in a patient comprising positioning an orthotic bone stimulator which comprises a bone growth stimulator attached to a stabilization device such that the bone growth stimulator can be positioned directly adjacent to the bone fracture of the patient and applying the bone growth stimulator to the fractured bone.

18. The method of claim 17, wherein the fractured bone is one or more bones selected from the group consisting of vertebrae, skull, rib, fibula, tibia, femur, scaphoid, metacarpal, metatarsal, humerus, ulna, radius and pelvis.

19. The method of claim 18, wherein the fractured bone is one or more vertebrae.

20. The method of claim 19, wherein the one or more vertebrae are in the cervical region.

21. The method of claim 17, wherein the stabilization device is a device for treating a compression fracture.

22. The method of claim 17, wherein the stabilization device is selected from the group consisting of a halo, cervical collar, thoracic orthosis, lumbosacral orthosis, thoracolumbar orthosis, and peripheral extremity orthosis.

23. The method of claim 17, wherein the bone growth stimulator is an ultrasonic bone stimulator.

24. The method of claim 17, wherein the bone growth stimulator is an electrical bone stimulator.

25. The method of claim 17, wherein the patient sufferers from one or more of tabes dorsalis, peroneal muscular atrophy, malignant neoplasm, diabetes, obesity, alcohol-induced mental disorders, alcohol dependence syndrome, tobacco use disorder, syringomyelia, syringobulbia, renal disease, arthrodesis status, or a mechanical complication due to an internal orthopedic device, implant, or graft.

26. The method of claim 25, wherein the mechanical complication of internal orthopedic device, implant, or graft is a spinal fusion failure.

27. A method for the prevention and/or treatment of osteoporotic bone loss in a patient comprising positioning an orthotic bone stimulator which comprises a bone growth stimulator attached to a stabilization device such that the bone growth stimulator can be positioned directly adjacent to a desired treatment site of the patient and applying the bone growth stimulator to the desired treatment site.

28. The method of claim 27, wherein the desired treatment site is one or more bones selected from the group consisting of vertebrae, skull, rib, fibula, tibia, femur, scaphoid, metacarpal, metatarsal, humerus, ulna, radius and pelvis.

29. The method of claim 28, wherein the desired treatment site is one or more vertebrae.

30. The method of claim 29, wherein the one or more vertebrae are in the cervical region.

31. The method of claim 27, wherein the desired treatment site is the pelvis.

32. The method of claim 27, wherein the stabilization device is selected from the group consisting of a halo, cervical collar, thoracic orthosis, lumbosacral orthosis, thoracolumbar orthosis, and peripheral extremity orthosis.

33. The method of claim 27, wherein the bone growth stimulator is an ultrasonic bone stimulator.

34. The method of claim 27, wherein the bone growth stimulator is an electrical bone stimulator.
